(19) United States
(12) Patent Application Publication
(10) Pub. No.: US 2005/0283206 A1
(43) Pub. Date: Dec. 22, 2005

(54) Skin electrodes with design thereon

(75) Inventors: Christian Hunt, McLean, VA (US); Ara Boghosian, Alexandria, VA (US)

Correspondence Address: GREENBLUM & BERNSTEIN, P.L.C., 1950 ROLAND CLARKE PLACE, RESTON, VA, 20191, US

(73) Assignee: Care Rehab and Orthopedic Products, Inc., McLean, VA (US)

(21) Appl. No.: 11/213,732

(22) Filed: Aug. 30, 2005

Related U.S. Application Data
(63) Continuation of application No. 10/347,764, filed on Jan. 22, 2003.

(51) Int. Cl. A61N 1/18

(52) U.S. Cl. 607/48

(57) ABSTRACT

Method of performing an orthopedic procedure using an orthopedic device and an electrode comprising a body portion having a first surface and indicia located on a side opposing the first surface. The method includes arranging the first surface of the electrode on a patient's skin during an orthopedic procedure so that the indicia provides for visual stimulation during the orthopedic procedure. This abstract is not intended to limit the invention in any way.

### CROSS-REFERENCE TO RELATED APPLICATIONS

The present application is a continuation of U.S. application Ser. No. 10/347,764 filed Jan. 22, 2003, the disclosure of which is expressly incorporated by reference herein in its entirety.

### 1. FIELD OF THE INVENTION

The present invention generally relates to electrodes having a design on an outer surface, and more particularly, to skin electrodes used with orthopedic devices for biofeedback or muscle stimulation having an outer surface with a design or indicia thereon.

### 2. DESCRIPTION OF THE PRIOR ART

Many types of medical devices use electrodes such as electrocardiograms, EKGs as well as orthopedic devices. These electrodes may be skin electrodes which have an adhesive layer for attaching to a patient's skin at a certain body location. For example, in EKG applications, the electrodes are attached to strategic points on a patient's body in order to detect electrical signals produced by the heart.

In orthopedic applications, the electrodes may be used for either muscle stimulation or biofeedback purposes. In these applications, the electrodes have terminals in which wires are attached thereto. The wires are also in electrical communication with the electrical or orthopedic device. In one example, the electrodes are capable of detecting the natural human body electrical signals generated by the muscles and transmit these signals to the orthopedic device. This would be equivalent to a biofeedback device. In another application, the electrodes may transfer electrical signals generated from the electrical device to the muscles. In this latter application, the transfer of the electrical signals from the orthopedic device will cause the patient's muscles to flex providing orthopedic benefits. This would be very similar to a muscle stimulation application.

Many types of devices in the industry are designed to gauge or measure certain responses. By way of example, U.S. Pat. No. 6,360,615 shows a position/movement sensitive effect emitting strain gauge device. This device includes a responsive portion, such as an electrically conductive fabric, that can be stretched or relaxed. In the stretched state, the electrical properties may be changed, such as resistance, and detected by a regulating circuit. In this situation, the regulating circuit sends a signal that depends on the detected electrical property to an effect-emitting component. This effect-emitting component may provide sound or light emission in response to such detected electrical property. In one application, the effect is a change in a facial expression in response to movement of the patient.

In other applications, for example, U.S. Pat. No. 6,455,752, a kit contains components for making decorated adhesive bandages. The kit comprises at least one adhesive bandage and at least one decorative element selected from adhesive stickers and adhesive tattoos. U.S. Pat. No. 4,285,338, similarly, discloses an adhesive bandage for covering and protecting a wound. This bandage contains an exterior illustration or may be irregularly formed to simulate a face or character. However, in both of these references, it is noted that the bandages are merely provided for covering wounds and are not used in any other type of application, and hence have a very limited application.

### SUMMARY OF THE INVENTION

It is an object of the present invention to provide a design on a surface of a skin electrode.

It is another object of the present invention to provide a design or other indicia on a surface of the skin electrode in order to provide a visually pleasing effect to the user during an orthopedic procedure.

It is still another object of the present invention to provide a design or other indicia on the skin electrode in order to divert the patient's attention during the orthopedic procedure.

In a first aspect of the invention, an electrode is provided which is used with an orthopedic device. The electrode includes a body portion having an adhesive surface and a conductive material, the adhesive surface being adapted to adhere to a user's skin during an orthopedic procedure. The electrode further includes indicia located on a side opposing the adhesive surface. The indicia provides for visual stimulation during the orthopedic procedure used with the orthopedic device.

In other aspects of the invention, the orthopedic device is one of a muscle stimulation, electrotherapy, inferential stimulation, and EMG device. Also, the electrode is preferably a skin electrode and may be of any geometrical shape. The body portion includes three layers which include, for example, (i) a conductive gel layer, (ii) a conductive material layer, and (iii) an outer surface layer including the indicia thereon.

In another aspect of the present invention, an orthopedic device used in orthopedic procedures is provided. The orthopedic device includes an orthopedic unit and a skin electrode. The skin electrode includes a body portion having an adhesive surface and a conductive material. The adhesive surface is adapted to adhere to a user's skin during an orthopedic procedure. Indicia is located on a side opposing the adhesive surface. The indicia provides for visual stimulation during the orthopedic procedure used with the orthopedic device. A wire is in electrical communication with the orthopedic unit and the skin electrode. The wire is adapted for (i) transmitting an electrical signal generated by the muscles of a patient to the orthopedic unit or (ii) transferring electrical signals generated from the orthopedic unit to the patient's muscles causing flexing of the muscle. In embodiments, the orthopedic unit is one of a muscle stimulation, electrotherapy, inferential stimulation, and EMG device. The skin electrode may be substantially a square shape, a round shape, and an octagonal shape.

In another aspect of the present invention, there is provided a method of performing an orthopedic procedure using an orthopedic device and an electrode comprising a body portion having an adhesive surface, a conductive material, and indicia located on a side opposing the adhesive surface. The method comprises adhering the adhesive surface of the electrode to a patient's skin during an orthopedic procedure so that the indicia provides for visual stimulation during the orthopedic procedure.

In another aspect of the present invention, the orthopedic procedure includes one of muscle stimulation, electrotherapy, inferential stimulation, and EMG.

In another aspect of the present invention, the indicia is a design, lettering, photograph or an action figure.

In another aspect of the present invention, the method further includes connecting the electrode to the orthopedic device via a wire.

In another aspect of the present invention, there is provided a method of performing an orthopedic procedure using an orthopedic unit and an orthopedic procedure skin electrode comprising a body portion having an adhesive surface, a conductive material, and indicia located on a side opposing the adhesive surface. The method comprises adhering the adhesive surface of the orthopedic procedure skin electrode to a patient's skin during an orthopedic procedure so that the indicia provides for visual stimulation during the orthopedic procedure and connecting, with a wire, the orthopedic procedure skin electrode to the orthopedic unit. The wire is adapted for one of transmitting an electrical signal generated by the patient's muscle to the orthopedic unit and transferring electrical signals generated from the orthopedic unit to the patient's muscle thereby causing flexing of the patient's muscle.

In another aspect of the present invention, the orthopedic procedure includes muscle stimulation, electrotherapy, inferential stimulation, and EMG.

In another aspect of the present invention, there is provided a method of stimulating a patient's muscle using an orthopedic device and an electrode comprising a body portion having a first surface, a conductive material, and indicia located on a side opposing the first surface, wherein the method comprises connecting the first surface of the electrode to a patient's skin during stimulation of the patient's muscle.

In another aspect of the present invention, there is provided a method of performing an orthopedic procedure using an orthopedic device and an electrode comprising a body portion having a first surface and indicia located on a side opposing the first surface, wherein the method comprises arranging the first surface of the electrode on a patient's skin during an orthopedic procedure so that the indicia provides for visual stimulation during the orthopedic procedure.

### BRIEF DESCRIPTION OF THE DRAWINGS

The foregoing and other objects, aspects and advantages will be better understood from the following detailed description of embodiments of the invention with reference to the drawings, in which:

FIG. 1 shows a plan view of an electrode of the present invention;

FIG. 2 shows the cross-sectional view of the electrode of the present invention; and

FIG. 3 shows a perspective view of an orthopedic assembly using the electrode of the present invention.

### DETAILED DESCRIPTION OF EMBODIMENTS OF THE INVENTION

The present invention is directed to electrodes used in muscle stimulation or biofeedback orthopedic devices. More particularly, the present invention is directed to skin electrodes for use with muscle stimulation, electrotherapy, inferential stimulation, EMG (biofeedback) or other similar orthopedic devices. The electrodes of the present invention are preferably skin electrodes and typically include an adhesive and/or sensing or emitting layer on a first surface and a design on a second surface of the electrode. The adhesive layer is designed to adhere the electrode to the patient, while the sensing or emitting device is used to, respectively, sense biofeedback responses or emit electrical stimulation to the patient's muscles for orthopedic applications. A terminal and wire assembly is provided for connecting the electrode to an orthopedic device for measurement of the biofeedback or for providing electrical stimulation to the muscles of the patient.

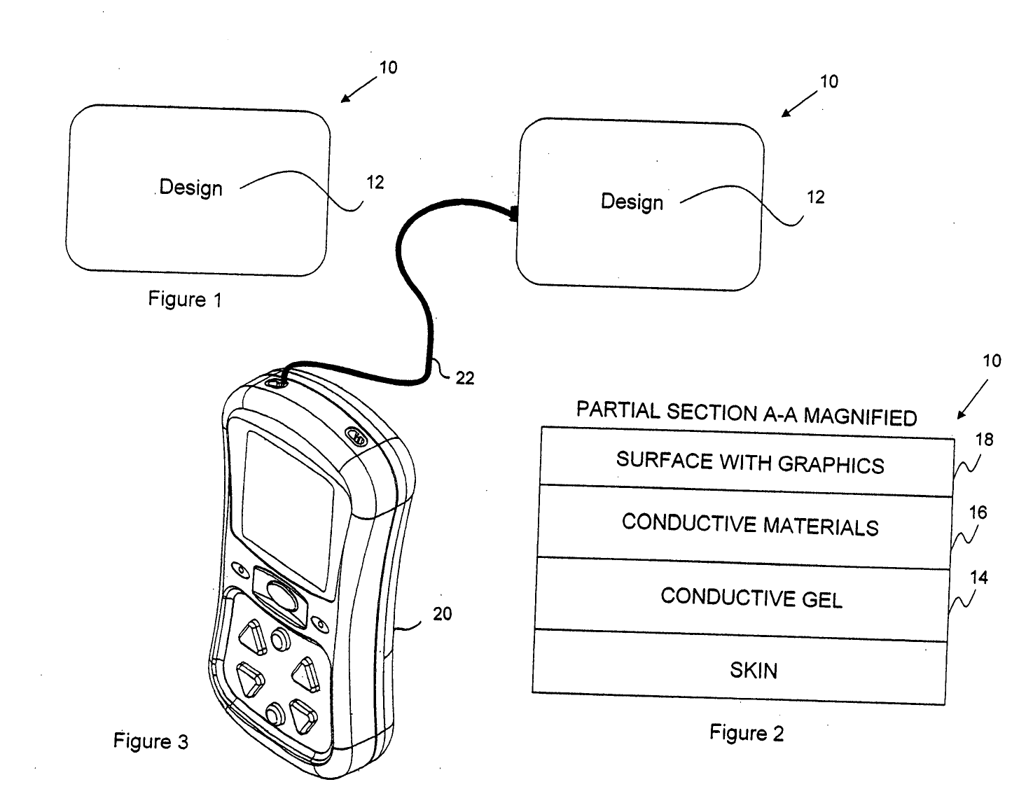

Now referring to FIG. 1, an electrode of the present invention is shown. The electrode is generally depicted as reference numeral 10 and includes indicia 12 thereon. This indicia 12 may be any type of character, lettering, photograph, action figure or any other type of design or the like. In any of these applications, the indicia is a visual effect which provides for entertainment and visual stimulation to the patient. This indicia may be helpful for diverting a patient's attention away from the orthopedic procedure, thus making it a more pleasant experience. This indicia may also be applicable, for example, when used with children in order to allow them to focus their attention on the design of the electrode. In essence, it is contemplated that the use of such indicia on the electrode may assist in providing for a “positive” experience to the patient while undergoing an orthopedic procedure such as electrical simulation. In preferred embodiments, the electrode 10 is a skin electrode.

FIG. 2 shows a cross-sectional view of the electrode of the present invention. As seen, the electrode 10 includes three surfaces or layers 14, 16, 18. In embodiments, the surface 14 is a conductive gel surface and surface 16 is a conductive material surface. The conductive material surface 16 may be connected to a wiring or electrical terminal in order to provide simulation or biofeedback, as discussed below. The outer surface 18 is the indicia surface which includes any type of design or other indicia. The conductive material surface 16 may be sandwiched between the conductive gel surface 14 and the outer surface 18. In an embodiment, the conductive gel and conductive material surfaces may be a single surface or layer. The conductive gel surface 14 is used to place the electrode on a patient's skin. The electrode of the present invention may be any shape and should not be limited to the substantially square shape shown herein. For example, the electrode of the present invention may be round, rectangular, octagonal or any other geometrical shape.

FIG. 3 shows the electrode of the present invention used with an orthopedic device. The orthopedic device generally depicted as reference numeral 20 is, in one application, a handheld orthopedic device which is in electrical communication with the electrode 10 via a wire 22. The orthopedic device 20 may be any one of a muscle stimulation, electrotherapy, inferential stimulation or EMG device. The wire 22 is preferable connected to a terminal 24 of the electrode. The orthopedic device is not limited to a handheld device. The wire is provided for detecting the natural human body electrical signals generated by the muscles and transmitting these signals to the orthopedic device. Likewise, the wires may transfer electrical signals generated from the orthopedic device to the muscles to cause the patient's muscles to flex.

Other aspects and features of the present invention can be obtained from a study of the drawings, the disclosure, and the appended claims.

1. A method of performing an orthopedic procedure using an orthopedic device and an electrode comprising a body portion having an adhesive surface, a conductive material, and indicia located on a side opposing the adhesive surface, the method comprising;
adhering the adhesive surface of the electrode to a patient's skin during an orthopedic procedure so that the indicia provides for visual stimulation during the orthopedic procedure.

2. The method of claim 1, wherein the orthopedic procedure includes one of muscle stimulation, electrotherapy, inferential stimulation, and EMG.

3. The method of claim 1, wherein the indicia is a design, lettering, photograph or an action figure.

4. The method of claim 1, further comprising connecting the electrode to the orthopedic device via a wire.

5. A method of performing an orthopedic procedure using an orthopedic unit and an orthopedic procedure skin electrode comprising a body portion having an adhesive surface, a conductive material, and indicia located on a side opposing the adhesive surface, the method comprising:
adhering the adhesive surface of the orthopedic procedure skin electrode to a patient's skin during an orthopedic procedure so that the indicia provides for visual stimulation during the orthopedic procedure; and
connecting, with a wire, the orthopedic procedure skin electrode to the orthopedic unit,
wherein the wire is adapted for one of transmitting an electrical signal generated by the patient's muscle to the orthopedic unit and transferring electrical signals generated from the orthopedic unit to the patient's muscle thereby causing flexing of the patient's muscle.

6. The method of claim 5, wherein the orthopedic procedure includes muscle stimulation, electrotherapy, inferential stimulation, and EMG.

7. The method of claim 5, wherein the indicia is a design, lettering, photograph or an action figure.

8. A method of stimulating a patient's muscle using an orthopedic device and an electrode comprising a body portion having a first surface, a conductive material, and indicia located on a side opposing the first surface, the method comprising;
connecting the first surface of the electrode to a patient's skin during stimulation of the patient's muscle.

9. The method of claim 8, further comprising providing, with the indicia, visual stimulation to the patient.

10. The method of claim 8, wherein the indicia is a design, lettering, photograph or an action figure.

11. The method of claim 8, further comprising connecting the electrode to the orthopedic device via a wire.

12. The method of claim 8, wherein the connecting comprises adhering the first surface of the electrode to the patient's skin during stimulation of the patient's muscle.

13. A method of performing an orthopedic procedure using a system consisting of an orthopedic device and an electrode connected to the orthopedic device wherein the electrode comprises a body portion having a first surface and indicia located on a side opposing the first surface, the method comprising;
arranging the first surface of the electrode on a patient's skin during an orthopedic procedure so that the indicia provides for visual stimulation during the orthopedic procedure.

14. The method of claim 13, wherein the orthopedic procedure includes one of muscle stimulation, electrotherapy, inferential stimulation, and EMG.

15. The method of claim 13, wherein the indicia is a design, lettering, photograph or an action figure.

16. The method of claim 13, further comprising connecting the electrode to the orthopedic device via a wire.

17. The method of claim 13, wherein the arranging comprises connecting the first surface of the electrode to the patient's skin.

18. The method of claim 13, wherein the arranging comprises adhering the first surface of the electrode to the patient's skin.
